(19) United States
(12) Patent Application Publication
(10) Pub. No.: US 2024/0041716 A1
(43) Pub. Date: Feb. 8, 2024

(54) GELLED AQUEOUS COMPOSITION COMPRISING A HEAVY-GRADE MAGNESIUM SALT

(71) Applicant: L'OREAL, Paris (FR)

(72) Inventors: Laura JAYAWARDENE, Chevilly Larue (FR); Claudia BARBA, Chevilly Larue (FR)

(73) Assignee: L'OREAL, Paris (FR)

(21) Appl. No.: 18/258,503

(22) PCT Filed: Dec. 21, 2021
(86) PCT No.: PCT/EP2021/087103

§ 371 Date: Jun. 20, 2023

(30) Foreign Application Priority Data
Dec. 22, 2020 (FR) FR2013915

(51) Int. Cl. A61K 8/19; A61Q 15/00

(52) U.S. Cl. CPC A61K 8/19; A61Q 15/00

(57) ABSTRACT

The present invention relates to a composition comprising —a gelled aqueous phase, —at least 1% by weight of active material of magnesium salt(s) having a poured bulk density of greater than 450 g/L, relative to the total weight of the composition, and —at least one glycol derivative, characterized in that the glycol derivative is chosen from propylene glycol, butylene glycol and mixtures thereof. The invention also relates to a cosmetic process for treating body odour associated with human perspiration, notably underarm odour, and optionally human perspiration, which consists in applying to the surface of the skin, in particular using a roll-on, an effective amount of said cosmetic composition.

The present invention relates to the field of compositions, notably cosmetic compositions, preferably deodorant compositions.

More particularly, the invention relates to the field of caring for and the hygiene of the skin, in particular bodily skin.

The present invention also relates to a process for the cosmetic treatment of the skin, and also to a cosmetic process for treating the body odour associated with human perspiration, notably underarm odour, and optionally human perspiration.

### TECHNICAL FIELD

In the cosmetics field, it is well known to use, in topical application, deodorant products containing active substances of antiperspirant type or of deodorant type for reducing or even preventing body odour, notably underarm odour, which is generally unpleasant.

Eccrine or apocrine sweat generally has little odour when it is secreted. It is its degradation by bacteria via enzymatic reactions which produces malodorous compounds. Deodorant active agents thus have the function of reducing or preventing the formation of unpleasant odours. This aim can be achieved notably by means of deodorant and/or antiperspirant activity.

### PRIOR ART

The various systems proposed hitherto may be grouped into major families.

A first family concerns unpleasant-odour absorbers. These absorbers “capture” or reduce the volatility of the odorous compounds.

Bactericidal substances which are preferably selective for the strains responsible for odours, or which limit the growth of the bacteria, are also known. Among the bactericidal substances which destroy the resident bacterial flora, the one most commonly used is Triclosan (2,4,4′-trichloro-2′-hydroxydiphenyl ether). Among the substances which reduce the growth of bacteria, mention may be made of transition metal-chelating agents such as EDTA or DPTA.

Substances which block the enzymatic reactions responsible for the formation of odorous compounds are also known, notably arylsulfatase, 5-lipoxygenase, aminoacylase or β-glucuronidase inhibitors.

Deodorant activity may also be obtained by neutralization of the volatile compounds responsible for the odour.

Finally, aluminium and/or zirconium salts are also used as antibacterial agents. These salts play a direct role on the deodorant efficacy by reducing the number of bacteria responsible for the degradation of sweat.

However, these various treatments applied to the skin of the armpits have a tendency to cause skin impairments.

Deodorant products are generally available in the form of roll-ons, tubes, sticks, aerosols or sprays.

The search for alternative solutions to the use of aluminium salts, or at least the search for solutions for limiting or even eliminating the use of these aluminium salts, has intensified. However, the deodorant products arising from this research often have the drawback of being less efficient in odour terms than products containing aluminium salts, and usually contain active agents that may be irritant, such as alcohol or essential oils.

In other words, these active agents have the drawback of giving rise to discomfort at the time of application, in particular after shaving the armpits.

The application of deodorant products using a roll-on or a ball applicator is particularly appreciated by consumers.

Compositions using magnesium salts as neutralizers or odour scavengers which make it possible to transform or to trap the volatile molecules responsible for the unpleasant odours have been formulated in the past. However, anti-odour performance equivalent to that of compositions comprising aluminium salts has not been achieved to date, notably for reasons of unfortunate competition between the volatile molecules responsible for the unpleasant odours and compounds present in the formulations or else for reasons of lack of stability of the compositions containing them.

In addition, consumers are sensitive to the fact that the products proposed in the field of deodorant compositions should not leave visible and unaesthetic marks, in particular white marks under the armpits and on clothing.

In order notably to overcome the problem of marks, deodorant compositions containing oils with a refractive index close to those of aluminium salts have been developed. The role of such oils is to reduce the whitish appearance of antiperspirant compositions when they are applied to the skin and, consequently, to make the marks on clothing less white.

However, such deodorant compositions have the drawback of giving the skin, notably on the armpits, an oily feel that is unpleasant for the user, and do not make it possible to limit the transfer of deodorant products from the skin onto clothing.

Moreover, nowadays, consumers are constantly in search of products whose effects on the environment and on health are reduced. In this respect, formulations comprising the least possible amount of ingredients are increasingly favoured.

### DESCRIPTION OF THE INVENTION

In particular, there is a need for roll-on deodorant compositions comprising at least one magnesium salt which has an improved anti-odour effect, in particular at least just as satisfactory as the effect obtained by applying a composition comprising aluminium salts, making it possible to overcome the occurrence of visible and unaesthetic marks, in particular white marks, under the armpits and on clothing, and moreover using the least possible amount of ingredients.

The aim of the present invention is to satisfy these needs.

### SUMMARY OF THE INVENTION

The present invention describes a composition comprising:
a gelled aqueous phase,
at least 1% by weight of active material of magnesium salt(s) having a poured bulk density of greater than 450 g/L, relative to the total weight of the composition, and
at least one glycol derivative.

Thus, according to a first aspect, the present invention is directed towards a composition comprising:
a gelled aqueous phase,
at least 1% by weight of active material of magnesium salt(s) having a poured bulk density of greater than 450 g/L, relative to the total weight of the composition, and
at least one glycol derivative, characterized in that the glycol derivative is chosen from propylene glycol, butylene glycol and mixtures thereof.

Contrary to all expectation, the inventors have found that the combination of at least 1% by weight of active material of magnesium salt(s) having a poured bulk density of greater than 450 g/L, relative to the total weight of the composition, and of at least one glycol derivative, in particular in a content of greater than 15% by weight, relative to the total weight of the composition, makes it possible to achieve the abovementioned aims.

Thus, as emerges from the examples featured below, the compositions according to the invention have both very satisfactory anti-odour efficacy and also sensory properties which also meet consumer expectations.

The compositions according to the invention thus make it possible to provide compositions that are relatively simple in terms of the number of ingredients, which not only meet the anti-odour performance requirements but also offer cosmetic properties that are pleasant, whether on application or after application.

According to another of its aspects, a subject of the invention is also a process for the cosmetic treatment of body odour associated with human perspiration, notably underarm odour, and optionally human perspiration, comprising the step of applying an effective amount of a composition as defined above to a skin surface, in particular by means of a roll-on.

The process of the invention is particularly advantageous for treating body odour associated with underarm perspiration.

The compositions, notably the cosmetic compositions, according to the invention comprise a physiologically acceptable medium.

For the purposes of the present invention, the term “physiologically acceptable medium” is intended to denote a medium that is suitable for the administration of a composition to the skin.

A physiologically acceptable medium generally has no unpleasant odour or appearance, and is entirely compatible with topical administration to the skin. In the present case, where the composition is intended for application to the surface of the skin, such a medium is considered in particular to be physiologically acceptable when it does not cause any stinging, tautness or redness that is unacceptable to the user.

In particular, the composition is suitable for application to the surface of the skin. Thus, the physiologically acceptable medium is preferentially a cosmetically or dermatologically acceptable medium, i.e. a medium that has no unpleasant odour, colour or appearance, and that does not cause the user any unacceptable stinging, tautness or redness.

The composition may then comprise any constituent usually used in the envisaged application.

Needless to say, a person skilled in the art will take care to select this or these optional additional compound(s), and/or the amount thereof, such that the advantageous properties of the compounds according to the invention are not, or are not substantially, adversely affected by the envisaged addition.

Other characteristics, aspects and advantages of the invention will become apparent on reading the detailed description which follows.

In the context of the present invention, the term “free of” indicates that the composition comprises less than 0.5% by weight, or even less than 0.2% by weight, and even more particularly less than 0.1% by weight of the compound concerned, relative to the total weight of the composition.

### DETAILED DESCRIPTION

### Magnesium Salt

Magnesium salts, and in particular magnesium oxide, are efficient for combating body odour. The higher the concentration of active agent, the more efficient will be the product. Thus, on account of their high basicity, magnesium salts, which in particular have a pH of greater than 10 in solution in water, have antibacterial action and in addition are capable of neutralizing the malodorous compounds of sweat such as the short-chain carbon-based volatile compounds of the acid family.

However, the difficulty of using this active agent is that of finding an equilibrium between the efficacy and its bleaching power on the skin and on clothing.

In the context of the present invention, the composition comprises at least 1% by weight of magnesium salt having a poured bulk density of greater than 450 g/L, relative to the total weight of the composition. This is referred to as a heavy-grade magnesium salt, as detailed hereinbelow.

The inventors have in fact found, surprisingly, as illustrated in the examples that follow, that the use of a heavy-grade magnesium salt makes it possible to significantly reduce or even to eliminate visible and unaesthetic marks, notably white marks, following the application of the composition under the armpits and/or on clothing. It has moreover been found that the choice of such a heavy-grade magnesium salt in comparison with a light-grade magnesium salt generally makes it possible to obtain a less viscous composition and to obtain a finer film on application. In other words, the use of the heavy-grade magnesium salt is very advantageous in aqueous formulation. The reason for this is that such a heavy grade magnesium salt does not give rise to substantial viscosity in formulation, allowing its use at a very high concentration while at the same time remaining applicable, more particularly in presentation forms such as roll-ons or ball applicators.

The magnesium salt is in particular chosen from magnesium oxide, magnesium carbonate, magnesium hydroxide, magnesium bicarbonate and mixtures thereof.

Magnesium oxide, magnesium carbonate or a mixture thereof, notably magnesium oxide, is more particularly used.

Magnesium oxide, magnesium hydroxide or a mixture thereof is more particularly alternatively used.

The magnesium salt, and in particular magnesium oxide, is in the form of a more or less dense white powder. As regards magnesium oxide, it is very sparingly soluble and transforms more or less quickly into its hydroxide form in the presence of water and can form a gel having a more or less thick texture. It has a very alkaline pH, in particular greater than 10.

The powder of a magnesium salt, in particular of magnesium oxide, thus comprises particles which may notably be characterized by their size, by their bulk density and also by their specific surface area.

The particles of a magnesium salt, in particular of magnesium oxide, may thus have, in the context of the present invention, a size (D50) which may be between 1 and 10 μm, in particular between 2 and 8 μm and even more particularly between 2 and 5 μm.

The particle size is typically measured by SLS particle size analysis (dispersion medium: water+1% SDS).

Conventionally, the magnesium oxide particles may have a poured bulk density which may be between 50 and 750 g/L, in particular between 80 and 750 g/L.

The poured bulk density may be measured at 20° C. on a 25 ml test tube in duplicate. The poured bulk density measurement is performed on a non-tapped sample. In other words, the poured bulk density is measured using a mass of sample in powder form, in a given volume, without any prior tapping.

It may be reminded that the poured and tapped bulk densities are classically measured to characterize the powder flowability and are typical measurements to characterize powders, especially in the pharmaceutical field as attested by various pharmacopoeias and also in the cosmetic field. Generally, poured bulk density may be determined by pouring a sample of known mass into a graduated cylinder, and measuring the volume occupied. Tapped bulk density may be determined by measuring the volume occupied by the sample after it is subjected to a prescribed amplitude and frequency of tapping over a prescribed time period using a tapped density tester.

In the framework of the present invention, the following method may be used:
1. A glass cylinder is weighed empty (M0 in g).
2. The powder is homogenized in its container by working it between the hands.
3. The glass cylinder is filled with the powder sample up to the 25 ml mark (V0 in ml), passing the sample through a funnel, without bumping or tapping the cylinder. The glass cylinder is weighed (M1 in g).

The poured bulk density in g/cm³ corresponds to the following formula:

$$\frac{M 1 - M 0}{V 0}$$

The protocol is repeated at least twice or until the mean value is less than 5%. A mean value is given and a difference of 10% is considered as acceptable.

Two different grades of magnesium oxide are thus distinguished according to the measured value of the poured bulk density, it being possible for both to be used in the context of the present invention.

More particularly, a “light magnesium salt”, in particular a “light-grade magnesium oxide”, denotes particles of a magnesium salt, in particular of magnesium oxide, having a poured bulk density of greater than or equal to 50 g/L and less than 250 g/L, in particular greater than or equal to 70 g/L and less than or equal to 200 g/L, and even more particularly greater than or equal to 80 g/L and less than or equal to 150 g/L.

In parallel, a “heavy magnesium salt”, in particular a “heavy-grade magnesium oxide”, which may be used in the context of the present invention, denotes particles of a magnesium salt having a poured bulk density of between 450 and 750 g/L, in particular between 500 and 700 g/L and even more particularly between 550 and 650 g/L.

The particles of a magnesium salt, in particular of magnesium oxide, may have a specific surface area (BET) which may be between 5 and 50 m²/g, in particular between 6 and 40 m²/g and even more particularly between 8 and 40 m²/g.

The specific surface area (BET) may conventionally be measured according to the following protocol: the particles are pretreated at 120° C. for 24 hours under nitrogen as absorption gas. According to a particular embodiment of the invention, the magnesium salt(s) with a poured bulk density of greater than 450 g/L used in the composition of the invention, in particular of magnesium oxide, have a specific surface area of less than 20 m²/g, in particular less than 15 m²/g, and even more particularly between 2 and 20 m²/g, or even between 4 and 15 m²/g. The specific surface area (BET) may conventionally be measured according to the following protocol: the particles are pretreated at 120° C. for 24 hours under nitrogen as absorption gas. Among the light-grade magnesium oxides, mention may notably be made of the magnesium oxides sold by the company UBE Industries under the names Ultra High Purity and Fine Magnesium Oxide 500A or the magnesium oxide sold by the company Dr. Paul Lohmann under the name Magnesium Oxide Extra Light.

Among the heavy-grade magnesium oxides that may be used in the context of the present invention, mention may notably be made of the magnesium oxides sold by the company Quaron/Magnesia or the company Dr. Paul Lohmann under the respective names Magnesia 23 and Magnesium Oxide Light. Mention may also be made in this respect of the magnesium oxide sold under the name Magnesia 22 by the company Dead Sea Bromine and Magnesium Oxide Heavy C46/124 by the company Dr. Paul Lohmann.

As magnesium dihydroxide that may be used in the context of the present invention, mention may notably be made of the magnesium hydroxide sold by the company Dr. Paul Lohmann.

According to a particular embodiment, the composition may comprise a mixture of a light-grade and heavy-grade magnesium salt, it being understood that the minimum content of heavy-grade magnesium salt is 1% by weight, relative to the total weight of the composition. Similarly, if the composition according to the present invention additionally comprises a light-grade magnesium salt, its content will preferentially not exceed 4% by weight, in particular 2% by weight, relative to the total weight of the composition, so as to avoid the appearance of the white marks mentioned above.

According to one embodiment, the magnesium salt(s) with a poured bulk density of greater than 450 g/L are present in a content ranging from 1% to 6% by weight, in particular from 1% to 4% by weight, even more particularly from 1% to 2% by weight of active material relative to the total weight of the composition.

It is understood that the content of magnesium salt(s) which would optionally be present in an additive as detailed below is not included in the content percentages indicated above.

### Glycol Derivative

For the purposes of the present invention, the term “hydrophilic gelling agent” means a compound that is capable of gelling the aqueous phase of the compositions according to the invention. The aqueous phase according to the present invention is at least gelled by the presence of at least one glycol derivative, it being understood that, as detailed below, the aqueous phase may also comprise an additional hydrophilic gelling agent.

The inventors have moreover found that the presence of at least one glycol derivative makes it possible to combat the potential instability, over time and with respect to temperature (4° C. and 45° C.), of a gel formed solely of water and of a magnesium salt. In particular, it makes it possible to combat the potential syneresis of said gel formed.

The glycol derivative is thus present in the aqueous phase of the composition.

For the purposes of the present invention, the term “glycol derivative” denotes a compound including 2 alcohol groups and including from 2 to 8 carbon atoms, in particular from 2 to 4 carbon atoms.

According to one aspect, the glycol derivative is chosen from propylene glycol and butylene glycol and mixtures thereof, and in particular is propylene glycol.

According to another aspect, the glycol derivative, and in particular the propylene glycol, the butylene glycol or mixtures thereof, is present in a content of at most 40% by weight, relative to the total weight of the composition, in particular of at most 35% by weight, for example at most 30% by weight, relative to the total weight of the composition.

Among the glycol derivatives, mention may be made of propylene glycol, 1,3-propanediol, butylene glycol and mixtures thereof.

The glycol derivative is more particularly chosen from propylene glycol, 1,3-propanediol and mixtures thereof.

According to a particular embodiment, when it is present, the butylene glycol is present in a content of less than 5% by weight, or even less than 4% by weight and even more particularly less than 3% by weight, relative to the weight of the composition.

According to a particular embodiment, propylene glycol is used, optionally as a mixture with butylene glycol.

According to one embodiment, the glycol derivative is present in a content ranging from 10% to 40% by weight, in particular from 15% to 40% by weight and even more particularly from 15% to 25% by weight relative to the total weight of the composition.

According to a particular embodiment of the invention, the composition comprises:
a gelled aqueous phase,
from 1% to 10% by weight, in particular from 1% to 5% by weight, of active material of magnesium salt(s) having a poured bulk density of greater than 450 g/L, relative to the total weight of the composition, and
from 10% to 30% by weight and even more particularly from 20% to 30% by weight of a glycol derivative, in particular of propylene glycol, optionally together with butylene glycol, relative to the total weight of the composition.

According to an even more particular embodiment, the magnesium salt, in particular magnesium oxide, has a specific surface area of less than 20 m²/g, in particular less than 15 m²/g, and even more particularly between 2 and 20 m²/g, or even between 4 and 15 m²/g. According to another even more particular embodiment, the composition also comprises at least one solid fatty alcohol as defined hereinbelow, in particular in a content of between 4% and 10% by weight, in particular between 4% and 8% by weight, relative to the total weight of the composition.

### Aqueous Phase

The aqueous phase of a composition according to the invention comprises water and optionally a water-soluble solvent.

In the present invention, the term “water-soluble solvent” denotes a compound that is liquid at room temperature and water-miscible (miscibility with water of greater than 50% by weight at 25° C. and atmospheric pressure).

The water-soluble solvents that may be used in the composition of the invention may also be volatile.

As indicated above, the aqueous phase of the composition according to the present invention is gelled at least by the presence of at least one glycol derivative as defined previously.

Among the water-soluble solvents that may be used in the composition in accordance with the invention, mention may notably be made of lower monoalcohols containing from 1 to 5 carbon atoms, such as ethanol and isopropanol.

According to one aspect, the composition according to the present invention comprises at least 60% by weight of water relative to the total weight of the composition, in particular at least 65% by weight, more particularly at least 70% by weight and even more particularly at least 75% by weight.

Preferably, a composition according to the invention comprises from 70% to 99% by weight, preferably from 75% to 95% by weight and more preferentially from 80% to 90% by weight of aqueous phase relative to the total weight of the composition.

In particular, a composition according to the invention comprises from 60% to 90% by weight, preferably from 65% to 80% by weight and more preferentially from 70% to 75% by weight of water relative to the total weight of the composition.

According to another embodiment variant, the aqueous phase of a composition according to the invention may comprise at least one C₂-C₃₂ polyol, other than the glycol derivatives defined previously.

For the purposes of the present invention, the term “polyol” should be understood as meaning any organic molecule including at least two free hydroxyl groups.

Preferably, a polyol in accordance with the present invention is present in liquid form at room temperature.

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols that are advantageously suitable for formulating a composition according to the present invention are those notably containing from 2 to 32 carbon atoms and preferably 3 to 16 carbon atoms.

Advantageously, the polyol may be chosen, for example, from pentaerythritol, trimethylolpropane, glycerol, polyglycerols, such as glycerol oligomers, for instance diglycerol, and polyethylene glycols, and mixtures thereof.

According to a particular embodiment of the invention, said polyol is chosen from glycerol.

### Additional Hydrophilic Gelling Agent

A composition according to the invention may comprise at least one additional hydrophilic gelling agent.

The additional hydrophilic gelling agent may be water-soluble or water-dispersible.

The additional hydrophilic gelling agent may be chosen from synthetic polymeric gelling agents, polymeric gelling agents that are natural or of natural origin, mixed silicates and fumed silicas, and mixtures thereof.

Preferably, the additional hydrophilic gelling agent may be chosen from synthetic polymeric gelling agents, polymeric gelling agents that are natural or of natural origin, and mixtures thereof.

More preferentially, the additional hydrophilic gelling agent may be chosen from synthetic polymeric gelling agents.

Polymeric Gelling Agents that are Natural or of Natural Origin

The polymeric hydrophilic gelling agents that are suitable for use in the invention may be natural or of natural origin.

For the purposes of the invention, the term “of natural origin” is intended to denote polymeric gelling agents obtained by modification of natural polymeric gelling agents.

These gelling agents may be particulate or non-particulate.

More specifically, these gelling agents fall within the category of polysaccharides.

In particular, the polysaccharides may be chosen from fructans, gellans, glucans, amylose, amylopectin, glycogen, pullulan, dextrans, celluloses, microcelluloses and derivatives thereof, in particular methylcelluloses, hydroxyalkylcelluloses, ethylhydroxyethylcelluloses and carboxymethylcelluloses, mannans, xylans, lignins, arabans, galactans, galacturonans, alginate-based compounds, chitin, chitosans, glucuronoxylans, arabinoxylans, xyloglucans, glucomannans, pectic acids and pectins, arabinogalactans, carrageenans, agars, glycosaminoglucans, gum arabics, tragacanth gums, ghatti gums, karaya gums, locust bean gums, galactomannans such as guar gums and nonionic derivatives thereof, in particular hydroxypropyl guar, and ionic derivatives thereof, biopolysaccharide gums of microbial origin, in particular scleroglucan or xanthan gums, mucopolysaccharides, and in particular chondroitin sulfates, and mixtures thereof, and preferably from biopolysaccharide gums of microbial origin.

### Synthetic Polymeric Gelling Agents

For the purposes of the invention, the term “synthetic” means that the polymer is neither naturally existing nor a derivative of a polymer of natural origin.

The synthetic polymeric hydrophilic gelling agent under consideration according to the invention may or may not be particulate.

For the purposes of the invention, the term “particulate” means that the polymer is in the form of particles, preferably spherical particles.

According to a particular embodiment of the invention, the composition contains less than 1% by weight of synthetic polymeric gelling agent, relative to the total weight of the composition, or even is free thereof.

### Fatty Phase

A composition according to the invention may also comprise at least one fatty phase.

In particular, a composition according to the invention comprises from 1% to 10% by weight, preferably from 1% to 8% by weight and more preferentially from 1% to 5% by weight of fatty phase relative to the total weight of the composition.

The fatty phase of the composition according to the invention may comprise oils.

The fatty phase may also comprise a lipophilic gelling agent.

### Oils

The term “oil” means a water-immiscible non-aqueous compound that is liquid at room temperature (20° C.) and at atmospheric pressure (760 mmHg).

An oily phase that is suitable for preparing the compositions, notably the cosmetic compositions, according to the invention may comprise hydrocarbon-based oils, silicone oils, fluoro oils or non-fluoro oils, or mixtures thereof.

The oils may be volatile or non-volatile.

They may be of animal, plant, mineral or synthetic origin.

The term “non-volatile” refers to an oil whose vapour pressure at room temperature and atmospheric pressure is non-zero and is less than 10⁻³ mmHg (0.13 Pa).

For the purposes of the present invention, the term “silicone oil” means an oil comprising at least one silicon atom, and notably at least one Si—O group.

The term “hydrocarbon-based oil” means an oil mainly containing hydrogen and carbon atoms.

The oils may optionally comprise oxygen, nitrogen, sulfur and/or phosphorus atoms, for example in the form of hydroxyl or acid radicals.

For the purposes of the invention, the term “volatile oil” means any oil that is capable of evaporating on contact with the skin in less than one hour, at room temperature and atmospheric pressure. The volatile oil is a volatile cosmetic compound, which is liquid at room temperature, notably having a non-zero vapour pressure, at room temperature and atmospheric pressure, notably having a vapour pressure ranging from 0.13 Pa to 40 000 Pa (10⁻³ to 300 mmHg), in particular ranging from 1.3 Pa to 13000 Pa (0.01 to 100 mmHg) and more particularly ranging from 1.3 Pa to 1300 Pa (0.01 to 10 mmHg).

### Volatile Oils

Preferably, the composition is free of petrochemistry-based oil and of silicone oil.

The volatile oils may be hydrocarbon-based oils or silicone oils.

Among the volatile hydrocarbon-based oils containing from 8 to 16 carbon atoms, mention may be made notably of branched C₈-C₁₆ alkanes, for instance C₈-C₁₆ isoalkanes (also known as isoparaffins), isododecane, isodecane, isohexadecane and, for example, the oils sold under the trade names Isopar or Permethyl, branched C₈-C₁₆ esters, for instance isohexyl neopentanoate, and mixtures thereof. In particular, the volatile hydrocarbon-based oil is chosen from volatile hydrocarbon-based oils containing from 8 to 16 carbon atoms and mixtures thereof.

Mention may also be made of volatile linear alkanes comprising from 8 to 16 carbon atoms, in particular from 10 to 15 carbon atoms and more particularly from 11 to 13 carbon atoms, for instance n-dodecane (C₁₂) and n-tetradecane (C₁₄) sold by Sasol under the respective references Parafol 12-97 and Parafol 14-97, and also mixtures thereof, the undecane-tridecane mixture, the mixtures of n-undecane (C₁₁) and of n-tridecane (C₁₃) obtained in Examples 1 and 2 of patent application WO 2008/155 059 from the company Cognis, and mixtures thereof.

Volatile silicone oils that may be mentioned include linear volatile silicone oils such as hexamethyldisiloxane, octamethyltrisiloxane, decamethyltetrasiloxane, tetradecamethylhexasiloxane, hexadecamethylheptasiloxane and dodecamethylpentasiloxane.

### Non-Volatile Oils

The non-volatile oils may notably be chosen from non-volatile hydrocarbon-based oils. Non-volatile hydrocarbon-based oils that may notably be mentioned include:
hydrocarbon-based oils of plant origin, synthetic ethers containing from 10 to 40 carbon atoms, such as dicaprylyl ether or polypropylene glycol stearyl ether, notably PPG-15 Stearyl Ether sold by the company Croda,
synthetic esters, for instance the oils of formula R₁COOR₂, in which R₁ represents a linear or branched fatty acid residue including from 1 to 40 carbon atoms and R₂ represents a hydrocarbon-based chain that is notably branched, containing from 1 to 40 carbon atoms, on condition that R₁+R₂ is greater than or equal to 10. The esters may notably be chosen from esters of alcohol and of fatty acid, for instance cetostearyl octanoate, esters of isopropyl alcohol, such as isopropyl myristate, isopropyl palmitate, ethyl palmitate, 2-ethylhexyl palmitate, isopropyl stearate, octyl stearate, hydroxylated esters, for instance isostearyl lactate, octyl hydroxystearate, alcohol or polyalcohol ricinoleates, hexyl laurate, neopentanoic acid esters, for instance isodecyl neopentanoate, isotridecyl neopentanoate, isononanoic acid esters, for instance isononyl isononanoate and isotridecyl isononanoate,
polyol esters and pentaerythritol esters, for instance dipentaerythrityl tetrahydroxystearate/tetraisostearate,
fatty alcohols that are liquid at room temperature, with a branched and/or unsaturated carbon-based chain containing from 12 to 26 carbon atoms, for instance 2-octyldodecanol, isostearyl alcohol and oleyl alcohol,
C₁₂-C₂₂ higher fatty acids, such as oleic acid, linoleic acid or linolenic acid, and mixtures thereof, and
carbonates, such as dicaprylyl carbonate, and also mixtures of these various oils.

In particular, the composition may also comprise at least one non-volatile oil, chosen in particular from non-volatile apolar hydrocarbon-based oils, non-volatile ester oils and mixtures thereof.

For the purposes of the present invention, the term “apolar oil” means an oil whose solubility parameter at 25° C., δₐ, is equal to 0 (J/cm³)^{1/2}.

The definition and calculation of the solubility parameters in the Hansen three-dimensional solubility space are described in the article by C. M. Hansen: “The three-dimensional solubility parameters”, J. Paint Technol. 39, 105 (1967).

According to this Hansen space:
δ_{D} characterizes the London dispersion forces derived from the formation of dipoles induced during molecular impacts;
δₚ characterizes the Debye interaction forces between permanent dipoles and also the Keesom interaction forces between induced dipoles and permanent dipoles;
δₕ characterizes the specific interaction forces (such as hydrogen bonding, acid/base, donor/acceptor, etc.); and
δₐ is determined by the equation: δₐ=(δp²+δh²)^{1/2}.
The parameters δₚ, δₕ, δ_{D} and δₐ are expressed in (J/cm³)^{1/2}.

In particular, the non-volatile apolar hydrocarbon-based oil is free of oxygen atoms.

Preferably, the non-volatile apolar hydrocarbon-based oil may be chosen from linear or branched hydrocarbons of mineral or synthetic origin. In particular, it may be chosen from:
liquid paraffin or derivatives thereof,
squalane,
liquid petroleum jelly,
polybutylenes, notably Indopol H-100 (molar mass or M_{w}=965 g/mol), Indopol H-300 (M_{w}=1340 g/mol) and Indopol H-1500 (M_{w}=2160 g/mol) sold or manufactured by the company Amoco,
polyisobutenes and hydrogenated polyisobutenes, notably Parleam® sold by the company Nippon Oil Fats, Panalane H-300 E sold or manufactured by the company Amoco (M_{w}=1340 g/mol), Viseal 20000 sold or manufactured by the company Synteal (M_{w}=6000 g/mol) and Rewopal PIB 1000 sold or manufactured by the company Witco (M_{w}=1000 g/mol),
decene/butene copolymers and polybutene/polyisobutene copolymers, notably Indopol L-14,
polydecenes and hydrogenated polydecenes, notably Puresyn 10 (M_{w}=723 g/mol) and Puresyn 150 (M_{w}=9200 g/mol) sold or manufactured by the company Mobil Chemicals,
and mixtures thereof.

Said non-volatile oil may also be an ester oil, in particular containing between 18 and 70 carbon atoms.

Examples that may be mentioned include monoesters, diesters or triesters.

The ester oils may notably be hydroxylated.

The non-volatile ester oil may preferably be chosen from:
monoesters comprising between 18 and 40 carbon atoms in total, in particular the monoesters of formula R₁COOR₂ in which R₁ represents a linear or branched fatty acid residue including from 4 to 40 carbon atoms and R₂ represents a hydrocarbon-based chain that is notably branched, containing from 4 to 40 carbon atoms, on condition that R₁+R₂ is greater than or equal to 18, for instance Purcellin oil (cetostearyl octanoate), isononyl isononanoate, C₁₂ to C₁₅ alkyl benzoate, 2-ethylhexyl palmitate, octyldodecyl neopentanoate, 2-octyldodecyl stearate, 2-octyldodecyl erucate, isostearyl isostearate, diisopropyl sebacate, 2-octyldodecyl benzoate, alcohol or polyalcohol octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate, 2-octyldodecyl myristate or 2-diethylhexyl succinate. Preferably, they are esters of formula R₁COOR₂ in which R₁ represents a linear or branched fatty acid residue including from 4 to 40 carbon atoms and R₂ represents a hydrocarbon-based chain that is notably branched, containing from 4 to 40 carbon atoms, R₁ and R₂ being such that R₁+R₂ is greater than or equal to 18. Preferably, the ester comprises between 18 and 40 carbon atoms in total. Preferred monoesters that may be mentioned include isononyl isononanoate, oleyl erucate and/or 2-octyldodecyl neopentanoate;
diesters, notably comprising between 18 and 60 carbon atoms in total and in particular between 18 and 50 carbon atoms in total. It is notably possible to use diesters of dicarboxylic acids and of monoalcohols, preferably such as diisostearyl malate, or glycol diesters of monocarboxylic acids, such as neopentyl glycol diheptanoate or polyglyceryl-2 diisostearate, notably such as the compound sold under the trade reference Dermol DGDIS by the company Alzo;
triesters, notably comprising between 35 and 70 carbon atoms in total, in particular such as triesters of a tricarboxylic acid, such as triisostearyl citrate, or tridecyl trimellitate, or glycol triesters of monocarboxylic acids such as polyglyceryl-2 triisostearate;
tetraesters, notably with a total carbon number ranging from 35 to 70, such as pentaerythritol or polyglycerol tetraesters of a monocarboxylic acid, for instance pentaerythrityl tetrapelargonate, pentaerythrityl tetraisostearate, pentaerythrityl tetraisononanoate, glyceryl tris(2-decyl)tetradecanoate, polyglyceryl-2 tetraisostearate or pentaerythrityl tetrakis(2-decyl)tetradecanoate;
polyesters obtained by condensation of unsaturated fatty acid dimer and/or trimer and of diol, such as those described in patent application FR 0 853 634, in particular such as of dilinoleic acid and of 1,4-butanediol. Mention may notably be made in this respect of the polymer sold by Biosynthis under the name Viscoplast 14436H (INCI name: dilinoleic acid/butanediol copolymer), or else copolymers of polyols and of dimer diacids, and esters thereof, such as Hailuscent ISDA;
esters and polyesters of diol dimer and of monocarboxylic or dicarboxylic acid, such as esters of diol dimer and of fatty acid and esters of diol dimer and of dicarboxylic acid dimer, in particular which may be obtained from a dicarboxylic acid dimer derived in particular from the dimerization of an unsaturated fatty acid notably of C₈ to C₃₄, notably of C₁₂ to C₂₂, in particular of C₁₆ to C₂₀ and more particularly of Cis, such as esters of dilinoleic diacids and of dilinoleic diol dimers, for instance those sold by the company Nippon Fine Chemical under the trade names Lusplan DD-DA5® and DD-DA7®;
vinylpyrrolidone/1-hexadecene copolymers, for instance the product sold under the name Antaron V-216 (also known as Ganex V216) by the company ISP (M_{w}=7300 g/mol);
hydrocarbon-based plant oils such as fatty acid triglycerides (which are liquid at room temperature), notably of fatty acids containing from 7 to 40 carbon atoms, such as heptanoic or octanoic acid triglycerides or jojoba oil; mention may be made in particular of saturated triglycerides such as caprylic/capric triglyceride, glyceryl triheptanoate, glyceryl trioctanoate, and C₁₈₋₃₆ acid triglycerides such as those sold under the reference DUB TGI 24 by Stéarinerie Dubois, and unsaturated triglycerides such as castor oil, olive oil, ximenia oil and pracaxi oil;
and mixtures thereof.

Preferably, a composition according to the invention comprises a non-volatile oil, and more preferentially polypropylene glycol stearyl ethers.

### Surfactant

When a fatty phase is present, the composition according to the invention may also comprise at least one surfactant.

The surfactants may be chosen from nonionic, anionic, cationic and amphoteric surfactants, and mixtures thereof. Reference may be made to Kirk-Othmer's Encyclopedia of Chemical Technology, Volume 22, pages 333-432, 3rd Edition, 1979, Wiley, for the definition of the emulsifying properties and functions of surfactants, in particular pages 347-377 of this reference, for anionic, amphoteric and nonionic surfactants.

### Nonionic Surfactant

The nonionic surfactants may notably be chosen from alkyl and polyalkyl esters of poly(ethylene oxide), oxyalkylenated alcohols, alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, gemini surfactants, and mixtures thereof.

Oxyalkylenated, in particular oxyethylenated and/or oxypropylenated, alcohols that are preferably used are those that may include from 1 to 150 oxyethylene and/or oxypropylene units, in particular containing from 20 to 100 oxyethylene units, in particular fatty alcohols, notably of C₈-C₂₄ and preferably of C₁₂-C₁₈; these fatty alcohols may or may not be ethoxylated, for instance stearyl alcohol ethoxylated with 20 oxyethylene units (CTFA name Steareth-20), for instance Brij® 78 sold by the company Uniqema, cetearyl alcohol ethoxylated with 30 oxyethylene units (CTFA name Ceteareth-30), cetearyl alcohol ethoxylated with 33 oxyethylene units (CTFA name Ceteareth-33 from the company SEPPIC) and the mixture of C₁₂-C₁₅ fatty alcohols including 7 oxyethylene units (CTFA name C₁₂₋₁₅ Pareth-7), for instance the product sold under the name Neodol 25-7® by Shell Chemicals; or in particular oxyalkylenated (oxyethylenated and/or oxypropylenated) alcohols containing from 1 to 15 oxyethylene and/or oxypropylene units, in particular ethoxylated C₈-C₂₄ and preferably C₁₂-C₁₈ fatty alcohols, such as stearyl alcohol ethoxylated with 2 oxyethylene units (CTFA name Steareth-2), for instance Brij® 72 sold by the company Uniqema.

Optionally polyoxyethylenated alkyl and polyalkyl esters of sorbitan that are preferably used include those with a number of ethylene oxide (EO) units ranging from 0 to 100. Examples that may be mentioned include sorbitan laurate 4 or 20 EO, in particular polysorbate 20 (or polyoxyethylene (20) sorbitan monolaurate) such as the product Tween® 20 sold by the company Uniqema, or polysorbate 60, sorbitan palmitate 20 EO, sorbitan isostearate, sorbitan stearate 20 EO, sorbitan oleate 20 EO, or else the Cremophor® products (RH 40, RH 60, etc.) from BASF. The mixture of sorbitan stearate and of sucrose cocoate, sold under the name Arlacel® 2121U-FL from Croda, may also be mentioned.

Alkyl and polyalkyl glucosides or polyglucosides that are preferably used are those containing an alkyl group including from 6 to 30 carbon atoms and preferably from 6 to 18 or even from 8 to 16 carbon atoms, and containing a glucoside group preferably comprising from 1 to 5 and notably 1, 2 or 3 glucoside units. The alkylpolyglucosides may be chosen, for example, from decylglucoside (alkyl-C₉/C₁₁-polyglucoside (1.4)), for instance the product sold under the name Mydol 10® by the company Kao Chemicals or the product sold under the name Plantacare 2000 UP® by the company Henkel and the product sold under the name Oramix NS 10® by the company SEPPIC; caprylyl/capryl glucoside, for instance the product sold under the name Plantacare KE 3711® by the company Cognis or Oramix CG 110® by the company SEPPIC; laurylglucoside, for instance the product sold under the name Plantacare 1200 UP® by the company Henkel or Plantaren 1200 N® by the company Henkel; cocoyl glucoside, for instance the product sold under the name Plantacare 818 UP® by the company Henkel; caprylyl glucoside, for instance the product sold under the name Plantacare 810 UP® by the company Cognis; the mixture of arachidyl glucosyl and behenyl alcohol and arachidyl alcohol, the INCI name of which is Arachidyl alcohol (and) behenyl alcohol (and) arachidyl glucoside, sold under the name Montanov® 202 by the company SEPPIC; and mixtures thereof.

The nonionic surfactants may also be chosen from hydrogenated castor oil, and more particularly an oxyethylenated hydrogenated castor oil, preferably comprising between 20 and 70 mol of ethylene oxide. More preferentially, said derivative is PEG-30 hydrogenated castor oil, PEG-40 hydrogenated castor oil or PEG-60 hydrogenated castor oil and decylglucoside from the company BASF, or caprylyl/capryl glycoside (Oramix CG 110L from SEPPIC).

### Anionic Surfactant

The anionic surfactants may be chosen from alkyl ether sulfates, carboxylates, amino acid derivatives, sulfonates, isethionates, taurates, sulfosuccinates, alkylsulfoacetates, phosphates and alkyl phosphates, polypeptides, metal salts of C₁₀-C₃₀ and notably C₁₆-C₂₅ fatty acids, in particular metal stearates and behenates, and mixtures thereof.

### Cationic Surfactant

The cationic surfactants may be chosen from alkylimidazolidiniums, such as isostearyl ethylimidonium ethosulfate, ammonium salts such as (C₁₂₋₃₀-alkyl)-tri(C₁₋₄-alkyl)ammonium halides such as N,N,N-trimethyl-1-docosanaminium chloride (or behentrimonium chloride).

### Amphoteric Surfactant

The compositions according to the invention may also contain one or more amphoteric surfactants, for instance N-acylamino acids such as N-alkyl aminoacetates and disodium cocoamphodiacetate, and amine oxides such as stearamine oxide, or alternatively silicone surfactants, for instance dimethicone copolyol phosphates such as the product sold under the name Pecosil PS 100® by the company Phoenix Chemical.

### Solid Fatty Alcohol

According to a particular embodiment of the invention, the composition also comprises a solid fatty alcohol.

The “solid fatty alcohols” are solid at room temperature (25° C.) and at atmospheric pressure (780 mmHg or 1 atm) and are water-insoluble, i.e. they have a solubility in water of less than 1% by weight and preferably less than 0.5% by weight.

The term “fatty alcohol” means a long-chain aliphatic alcohol comprising from 8 to 40 carbon atoms, preferably from 12 to 34 or even 12 to 30 carbon atoms, and comprising at least one hydroxyl group OH. These fatty alcohols are neither oxyalkylenated nor glycerolated.

Preferably, the solid fatty alcohols have the structure R—OH with R denoting a linear alkyl group, optionally substituted with one or more hydroxyl groups, comprising from 12 to 40, better still from 12 to 34 or even from 12 to 30 and most preferentially from 12 to 24 carbon atoms.

The solid fatty alcohols that may be used in the context of the invention are more particularly chosen from:
lauryl alcohol (1-dodecanol);
myristyl alcohol (1-tetradecanol);
cetyl alcohol (1-hexadecanol);
stearyl alcohol (1-octadecanol);
arachidyl alcohol (1-eicosanol);
behenyl alcohol (1-docosanol);
lignoceryl alcohol (1-tetracosanol);
ceryl alcohol (1-hexacosanol);
montanyl alcohol, (1-octacosanol);
myricyl alcohol, (1-triacontanol);
and mixtures thereof.

More particularly, the solid fatty alcohol is chosen from cetyl alcohol, stearyl alcohol, behenyl alcohol and mixtures thereof such as cetylstearyl alcohol or cetearyl alcohol. Cetearyl alcohol comprises a mixture of stearyl and cetyl alcohol, and is particularly suitable for the composition according to the present invention.

The fatty alcohols may be mixtures, which means that several species may coexist in a commercial product, notably species of different chain lengths, in the form of a mixture.

The solid fatty alcohol(s) according to the invention are preferably present in the composition in an amount ranging from 0.01% to 30% by weight, notably from 0.1% to 15% by weight, preferentially from 0.5% to 12% by weight, even better still from 2% to 12% by weight or even better from 4% to 10% by weight, relative to the total weight of the composition.

### Additives

The cosmetic compositions according to the invention may also comprise cosmetic adjuvants chosen from deodorant active agents, antioxidants, opacifiers, stabilizers, moisturizers, vitamins, bactericides, preserving agents, polymers, fragrances, thickeners or suspension agents or any other ingredient usually used in cosmetics for this type of application.

Needless to say, a person skilled in the art will take care to select this or these optional additional compounds such that the advantageous properties intrinsically associated with the composition in accordance with the invention are not, or are not substantially, adversely affected by the envisaged addition(s).

Preferably, a composition according to the invention is free of aluminium salts or complexes.

### Moisture Absorbers

It is also possible to add moisture absorbers, for instance perlites and preferably expanded perlites.

The cosmetic composition may comprise one or more moisture absorbers chosen from perlites.

Preferably, the cosmetic composition comprises one or more absorbers chosen from expanded perlites.

The perlites that may be used according to the invention are generally aluminosilicates of volcanic origin and have the composition:
70.0-75.0% by weight of silica SiO₂;
12.0-15.0% by weight of aluminium oxide Al₂O₃;
3.0-5.0% of sodium oxide Na₂O;
3.0-5.0% of potassium oxide K₂O;
0.5-2% of iron oxide Fe₂O₃;
0.2-0.7% of magnesium oxide MgO;
0.5-1.5% of calcium oxide CaO; and
0.05-0.15% of titanium oxide TiO₂.

The perlite is ground, dried and then calibrated in a first step. The product obtained, known as perlite ore, is grey-coloured and has a size of about 100 μm.

The perlite ore is then expanded (1000° C./2 seconds) to give more or less white particles. When the temperature reaches 850-900° C., the water trapped in the structure of the material evaporates and brings about the expansion of the material, relative to its original volume. The expanded perlite particles in accordance with the invention may be obtained via the expansion process described in patent U.S. Pat. No. 5,002,698.

Preferably, the perlite particles used will be milled; in this case, they are known as Expanded Milled Perlite (EMP). They preferably have a particle size defined by a median diameter D50 ranging from 0.5 to 50 μm and preferably from 0.5 to 40 μm. Preferably, the perlite particles used have a loose bulk density at 25° C. ranging from 10 to 400 kg/m³ (standard DIN 53468) and preferably from 10 to 300 kg/m³.

Preferably, the expanded perlite particles according to the invention have a water absorption capacity, measured at the wet point, ranging from 200% to 1500% and preferably from 250% to 800%.

The wet point corresponds to the amount of water which has to be added to 1 g of particle in order to obtain a homogeneous paste. This method is directly derived from the oil uptake method applied to solvents. The measurements are taken in the same manner by means of the wet point and the flow point, which have, respectively, the following definitions:
wet point: mass expressed in grams per 100 g of product corresponding to the production of a homogeneous paste during the addition of a solvent to a powder;
flow point: mass, expressed in grams per 100 g of product, at and above which the amount of solvent is greater than the ability of the powder to retain it. This is reflected by the production of a more or less homogeneous mixture which flows over the glass plate.

The wet point and the flow point are measured according to the following protocol:

Protocol for Measuring the Water Absorption

1) Equipment Used
Glass plate (25×25 mm)
Spatula (wooden shaft and metal part (15×2.7 mm))
Silk-bristled brush
Balance

2) Procedure

The glass plate is placed on the balance and 1 g of perlite particles is weighed out. The beaker containing the solvent and the sample liquid is placed on the balance. The solvent is gradually added to the powder, the whole being regularly blended (every 3 to 4 drops) by means of the spatula.

The mass of solvent needed to obtain the wet point is noted. Further solvent is added and the mass which makes it possible to reach the flow point is noted. The average of three tests will be determined.

The expanded perlite particles sold under the trade names Optimat 1430 OR or Optimat 2550 by the company World Minerals will be used in particular.

### Preserving Agent

According to one embodiment, the composition according to the invention may also comprise at least one preserving agent.

The preserving agent is a preserving agent commonly used in cosmetics. It may be chosen from the positive list contained in Annex V of (EC) Regulation No. 1223/2009, which specifies the list of preserving agents permitted in cosmetics. These ingredients have the function of stabilizing the formulation from a bacteriological viewpoint.

A composition according to the invention may comprise from 0.001% to 5% by weight and preferably from 0.1% to 1.5% by weight of preserving agent(s) relative to the total weight of the composition.

Preferably, the composition according to the invention is free of preserving agents. This embodiment may notably be performed when the composition has a pH of greater than 10.

### Presentation Forms

According to one embodiment, the composition according to the present invention is in the form of an aqueous gel or an aqueous dispersion of solid fatty alcohol, which is particularly useful for use using a roll-on or a ball applicator.

When a fragrance and/or the fatty alcohol is present, the presence of a surfactant may prove to be useful. In the case of this embodiment, the composition may thus have the appearance of a white milk.

The composition according to the present invention may have a large range of textures as a function of the contents of magnesium salts and of glycol derivative. In particular, the higher the content of glycol derivative, the thicker the texture.

Similarly, the inventors have found that the use of heavy-grade magnesium oxides may prove to be advantageous for obtaining more fluid compositions. In other words, for an equivalent content, heavy-grade magnesium oxides make it possible to obtain compositions that are more fluid than light-grade magnesium oxides. In other words, by modifying the grade of the magnesium oxide, it is possible to modify the texture of the composition.

According to a particular embodiment, the present invention relates to a composition in the form of an aqueous gel, comprising:
at least 1% by weight of magnesium oxide active material with a poured bulk density of greater than 450 g/L, relative to the total weight of the composition, the poured bulk density notably being able to be between 450 and 750 g/L, in particular between 500 and 700 g/L and even more particularly between 550 and 650 g/L, and
at least one glycol derivative, notably at least 10% by weight of a glycol derivative, in particular at least 10% by weight of propylene glycol, relative to the total weight of the composition.

### Viscosity

The viscosity of the composition is measured at room temperature (25° C.) using a viscometer RHEOMAT RM 200 equipped with a mobile No. 3 or 4 (M3, M4), the measurement being made after 10 minutes of rotation of the mobile in the product (time at the end of which we observe a stabilization of viscosity and speed of rotation of the mobile), at a shear of 200 s⁻¹. The results are given in UD (Unit of Deviation), then with the help of an Abacus transcribed in Poise then in Pa·s⁻¹.

Since the compositions are intended for application using a roll-on, they may have a viscosity at 25° C. and at 30 seconds ranging from 20 to 70 DU (spindle 2), in particular from 20 to 60 DU, even more particularly from 20 to 40 DU.

When they are in gel form, in particular in the absence of solid fatty alcohol and/or of fragrance and of a surfactant, the compositions may have a viscosity at 25° C. and at 30 seconds ranging from 20 to 50 DU (spindle 2).

In the form of an aqueous dispersion, the compositions may have a viscosity at 25° C. and at 30 seconds ranging from 50 to 90 DU (spindle 2).

The compositions according to the invention may be prepared by a person skilled in the art, according to the conventionally known methods.

According to a particular embodiment of the invention, the composition according to the present invention is transparent.

According to another embodiment, the invention also relates to a cosmetic process for treating body odour associated with human perspiration, notably underarm odour, and optionally human perspiration, which consists in applying to the surface of the skin, in particular using a roll-on, an effective amount of the cosmetic composition as described previously.

The present invention also relates to the use of a composition according to the invention for the cosmetic treatment of body odour associated with human perspiration, notably underarm odour, and optionally human perspiration.

Throughout the description, including the claims, the term “including a” should be understood as being synonymous with “including at least one”, unless otherwise specified. The terms “between . . . and . . . ”, “comprises from . . . to . . . ”, “formed from . . . to . . . ” and “ranging from . . . to . . . ” should be understood as being inclusive of the limits, unless otherwise specified.

The invention is illustrated in greater detail by the examples and FIGURES presented below. Unless otherwise indicated, the amounts shown are expressed as mass percentages.

### EXAMPLE

### Example I: Composition in Gel Form

The following compositions, in gel form, are prepared as described below with the proportions indicated in Table 2.

In Tables 2 and 3 below, the characteristics of the magnesium oxides used are as indicated in Table 1 below:

TABLE 1

| | Poured bulk | BET specific |
|---|---|---|
| Ingredient | density (g/L) | surface area (m²/g) |
| Light-grade magnesium oxide | ≈80 g/L | 30-40 |
| sold under the name | | |
| Magnesium Oxide Extra Light | | |
| by the company Dr. Paul | | |
| Lohmann-“MgO 1” | | |
| Heavy-grade magnesium | Between 500 and | 8-20 |
| oxide sold under the name | 750 g/L | |
| Magnesium Oxide Heavy | | |
| C46/124 by the company Dr. | | |
| Paul Lohmann-“MgO 2” | | |
| Heavy-grade magnesium | Between 500 and | 8-20 |
| oxide sold under the name | 750 g/L | |
| Magnesia 22 by the company | | |
| Dead Sea Bromine-“MgO 3” | | |

TABLE 2

| | | Comp. | | |
|---|---|---|---|---|
| Ingredient | A | A O.I. | B | C |
| Propylene glycol | 25.0 | 25.0 | 25.0 | 25.0 |
| Caprylyl glycol | | 0.5 | 0.5 | |
| “MgO 1” | | 2.0 | | |
| “MgO 2” | 2.0 | | | |
| “MgO 3” | | | 2.0 | 5.0 |
| Microcrystalline cellulose and cellulose | 2.0 | | | |
| gum sold under the name Avicel PC 591 | | | | |
| by the company FMC Corporation | | | | |
| Hydroxyethylcellulose sold under the | 0.25 | 0.25 | 0.35 | 0.25 |
| name Natrosol 250 HHR by the | | | | |
| company Ashland | | | | |
| Hydroxypropyl starch phosphate sold | | 2.5 | 2.5 | 2.25 |
| under the name Structure Zea by the | | | | |
| company AkzoNobel | | | | |
| PEG-60 Hydrogenated Castor Oil | 0.5 | 0.25 | 0.25 | 0.25 |
| (INCI) sold under the name Eumulgin | | | | |
| CO 60 by the company BASF | | | | |
| Vitamin | | | 0.01 | |
| Fragrance | 1.2 | 1.2 | 1.2 | 1.2 |
| Water | qs | qs | qs | qs |

### Preparation of the Compositions

The compositions are prepared as follows:
The water and the microcrystalline cellulose are placed in a tank with emulsification at high shear (3000 rpm) for 10 minutes,
The gelling agent is added and the mixture is heated to 60° C.,
The mixture is stirred for 20 minutes and the swelling of the gel is checked (absence of lumps),
The mixture is cooled to 35° C.,
The propylene glycol, the fragrance, the MgO and the surfactant are added,
The mixture is left stirring until homogenized,
Satisfactory dispersion of the MgO on spreading is checked on a black plate or a glass plate with a black background,
If grains of MgO powder are absent, the tank is emptied, otherwise vacuum emulsification is recommenced and the mixture is checked again.

Compositions A, B and C gave satisfactory anti-odour results, in particular in comparison with a product of the same presentation form containing 30% of aluminium salts (percentage of the starting material). This comparative formulation is considered to be very efficient against odours.

### Anti-Odour Performance Protocol

The performance was thus evaluated in an in vivo multi-application test by an expert panel. The test consists of a standardized evaluation of the intensities of unpleasant odour, of the fragrance and of the olfactory description of the unpleasant armpit odour by 10 panellists. 0.4 g of product is applied randomly to the armpits of 20 volunteers having strong underarm odour.

The olfactory intensity is evaluated on paper (grade from 0 to 100) and then input for the data processing into software on a continuous linear scale from “no odour” (0) to “extreme odour” (100) with a reference point at 50. The acquisition and data processing were performed by FIZZ® and XLStat®.

Thus, the combination of propylene glycol with high-grade magnesium oxide in a content of greater than 2% gives anti-odour performance that is just as high as that of a product containing 30% of aluminium salts, which are reputed to afford a very efficient anti-odour effect.

### Example II: Compositions in the Form of an Aqueous Dispersion Comprising a Solid Fatty Alcohol

The following compositions, in the form of an aqueous dispersion, according to the invention, are prepared as described below with the proportions indicated in Table 3. The compositions marked “0.1.” denote the compositions outside the invention.

TABLE 3

| | | | | Comp. B | Comp. F |
|---|---|---|---|---|---|
| Ingredient | D | E | B | O.I. | O.I. |
| Propylene glycol | 25.0 | | | | |
| Caprylyl glycol | | 0.4 | 0.4 | 0.4 | |
| Butylene glycol | | 3.0 | 3.0 | 3.0 | |
| “MgO 1” | | | | 6.0 | |
| “MgO 2” | 2.0 | | | | |
| “MgO 3” | | 6.0 | 15.0 | | |
| Aluminium chlorohydrate (Achieve | | | | | 30 (15% by |
| L1050) from the company | | | | | weight of |
| Gulbrandsen Technologies | | | | | active materials) |
| Microcrystalline cellulose and | 1.5 | | | | |
| cellulose gum sold under the name | | | | | |
| Avicel PC 611 by the company FMC | | | | | |
| Corporation | | | | | |
| PPG-15 Stearyl Ether sold uner the | | | 3.0 | | |
| name SP Arlamol PS15E VIT E | | | | | |
| MBAL-LQ-(RB) by the company | | | | | |
| Croda | | | | | |
| Perlite sold under the name Optimat | | | 1.0 | | |
| 2250 OR by the company World | | | | | |
| Minerals (Imerys) | | | | | |
| Dimethicone | | | 0.5 | | 0.5 |
| Ceteareth-33 sold under the name | 0.5 | 1.25 | 1.25 | 1.25 | 1.25 |
| Simulsol CS Ecailles by the company | | | | | |
| SEPPIC | | | | | |
| Cetearyl alcohol sold under the name | 2.0 | 3.0 | 3.0 | 3.0 | 2.5 |
| Lanette D by the company BASF | | | | | |
| Fragrance | 1.2 | 1.2 | | | |
| Water | qs | qs | qs | qs | qs |

### Preparation of the Compositions of the Examples

The compositions are prepared as follows:
The gelling agent is introduced into the water in a tank with a paddle stirrer
The mixture is stirred at high shear (3000 rpm)
The mixture is heated to 75° C. under vacuum, the fatty phase is added and the resulting mixture is emulsified for 10 minutes
The mixture is stirred with the paddle stirrer and cooled to a temperature of 35° C.,
The propylene glycol, the fragrance and the MgO are added,
The mixture is left to cool to 25° C.,
Satisfactory dispersion of the MgO on spreading is checked on a black plate or a glass plate with a black background,
If grains of MgO powder are absent, the tank is emptied, otherwise vacuum emulsification is recommenced and the mixture is checked again.

Compositions D, E and G gave satisfactory anti-odour results, in particular in comparison with a product of the same presentation form containing 30% of aluminium salts. This comparative formulation is considered to be very efficient against odours.

Finally, a comparison was performed between composition G (Example II) and comp. composition B (Example II) relative to the effect on marks.

### White Marks on a Support:

0.2 g of composition is applied and spread over an area of 4 cm by 4 cm on a flesh-coloured and black-coloured flexible support, known as “suplale”. Sais “suplale” is a fake leather material known as a flexible elastomer having properties next to the one of the skin. The deposit is dried in the open air for at least 4 hours. The white marks are then visualized on each support.

It was observed that the intensity of the white marks is less pronounced for composition G than for comp. composition B, according to the table of results collated in Table 4 below: the scale ranging from “−”: no marks, to “+++”: substantial presence of marks.

TABLE 4

| | D | E | G | Comp B O.I. | Comp F O.I. |
|---|---|---|---|---|---|
| White marks | +/− | + | ++ | +++ | — |

### Example III: Evaluation of the Deposition of Marks

An evaluation of consumer test type on a sample of nine women from 31 to 53 years old was performed in order to evaluate any deposition of marks.

These women had skin of phototype III to V and small armpits (xs-s size).

The armpits were washed, the two armpits were treated with, for the first, an application of composition B (Example I) or of comparative comp. A (Example I). The technician applied 0.4 g of product. The armpit was dried for 10 minutes in the open air.

A black T-shirt was worn for 5 hours: 35 minutes in a waiting room and then 4 hours 30 minutes under normal living conditions.

An evaluation was made by taking photographs at 45 minutes and then 5 hours after application to the armpits.

The white marks on the clothing were photographed 35 minutes after application and 5 hours after application.

### Formulations Tested

Composition B was evaluated in vivo and compared with a completely transparent reference, approved as mark-free, detailed in Table 5 below and with the composition comparative comp. A.

TABLE 5

| | Comparative composition- |
|---|---|
| Ingredient | evaluation deposition of marks |
| Fragrance | 1.2 |
| Propylene glycol | 15.0 |
| Silica silylate sold under the | 0.3 |
| name DOW Corning VM-2270 | |
| Aerogel Fine Particles (Dow | |
| Corning) | |
| Preserving agent | 0.5 |
| Caprylyl glycol | 0.5 |
| Xanthan gum | 0.39 |
| Capryloyl salicylic acid sold | 0.3 |
| under the name Mexoryl SAB | |
| by the company Noveal | |
| Dehydroxylated xanthan gum | 0.29 |
| sold under the name Amaze XT | |
| by the company AkzoNobel | |
| (Nouryon) | |
| Water | qs |

### Results

### 1. On the Armpits

As regards the composition comparative comp. A, a visible to very visible deposit is observed for seven out of eight individuals.

As regards composition B, absence of a deposit or a sparingly visible deposit is observed.

### 2. On a Black T-Shirt

The area of the marks is very large ˜20 cm² for comparative comp. A, ˜15 cm² for composition B, these marks being rather grey in colour, and the transparent comparative formulation (Table 4) indicated previously leaves few marks.

### CONCLUSION

The results show that the composition comparative comp. A gives a sparingly visible fine and/or powdery deposit covering all of the hairy area and/or the contour of the armpit. The deposit has a tendency to diminish on the skin in the course of the day. In contrast, for an equivalent concentration of MgO, composition B does not leave any marks on the skin.

As regards the marks on clothing, the marks were evaluated on a black T-shirt. The results show that composition B has a tendency to deposit fewer white marks on the black fabric than the composition comparative comp. A.

1: A composition comprising:
a gelled aqueous phase,
at least 1% by weight of active material of magnesium salt(s) having a poured bulk density of greater than 450 g/L, relative to the total weight of the composition, and
at least one glycol derivative,
wherein the glycol derivative is selected from the group consisting of propylene glycol, butylene glycol and mixtures thereof.

2: The composition according to claim 1 wherein the magnesium salt is selected from the group consisting of magnesium oxide, magnesium carbonate, magnesium hydroxide, magnesium bicarbonate, and mixtures thereof.

3: The composition according to claim 1, wherein the magnesium salt is magnesium oxide.

4: The composition according to claim 1, wherein the magnesium salt(s) with a poured bulk density of greater than 450 g/L are present in a content ranging from 1% to 6% by weight of active material relative to the total weight of the composition.

5: The composition according to claim 1, wherein the magnesium salt(s) with a poured bulk density of greater than 450 g/L have a poured bulk density of between 450 and 750 g/L.

6: The composition according to claim 1, wherein the magnesium salt(s) with a poured bulk density of greater than 450 g/L have a specific surface area of less than 20 m²/g.

7: The composition according to claim 1, wherein the glycol derivative is present in a content of at most 40% by weight, relative to the total weight of the composition.

8: The composition according to claim 1, wherein the glycol derivative is selected from the group consisting of propylene glycol, 1,3-propanediol, butylene glycol and mixtures thereof.

9: The composition according to claim 1, wherein the glycol derivative is present in a content ranging from 10% to 40% by weight relative to the total weight of the composition.

10: The composition according to claim 1, further comprising at least 60% by weight of water relative to the total weight of the composition.

11: The composition according to claim 1, having the form of an aqueous gel or an aqueous dispersion comprising a solid fatty alcohol.

12: The composition according to claim 11, wherein the solid fatty alcohol is selected from the group consisting of lauryl alcohol; myristyl alcohol; cetyl alcohol; stearyl alcohol; arachidyl alcohol; behenyl alcohol; lignoceryl alcohol; ceryl alcohol; montanyl alcohol; myricyl alcohol and mixtures thereof.

13: A cosmetic process for treating body odour associated with human perspiration, comprising applying to the surface of the skin, an effective amount of the cosmetic composition as defined according to claim 1.

14: The composition according to claim 1, wherein the magnesium salt(s) with a poured bulk density of greater than 450 g/L are present in a content ranging from 1% to 2% by weight of active material relative to the total weight of the composition.

15: The composition according to claim 1, wherein the magnesium salt(s) with a poured bulk density of greater than 450 g/L have a poured bulk density of between 550 and 650 g/L.

16: The composition according to claim 1, wherein the magnesium salt(s) with a poured bulk density of greater than 450 g/L between 2 and 20 m²/g.

17: The composition according to claim 1, wherein the magnesium salt(s) with a poured bulk density of greater than 450 g/L is magnesium oxide.

18: The composition according to claim 1, wherein the glycol derivative is present in a content ranging from 15% to 25% by weight, relative to the total weight of the composition.

19: The composition according to claim 1, wherein it comprises at least 75% by weight of water relative to the total weight of the composition.
